(19) United States
(12) Patent Application Publication
(10) Pub. No.: US 2026/0178638 A1
(43) Pub. Date: Jun. 25, 2026

(54) LARGE MODEL-BASED MEDICAL INFORMATION RECOMMENDATION METHOD, DEVICE, AND COMPUTER-READABLE STORAGE MEDIUM

(71) Applicant: VALUE TECH LIMITED, HONG KONG SAR (CN)

(72) Inventor: Kam Shing NG, HONG KONG SAR (CN)

(21) Appl. No.: 19/431,621

(22) Filed: Dec. 23, 2025

(30) Foreign Application Priority Data
Dec. 23, 2024 (CN) 2024119022519

(51) Int. Cl. G06F 16/334; G16H 10/60

(52) U.S. Cl. CPC G06F 16/3347; G16H 10/60

(57) ABSTRACT

A large model-based medical information recommendation method, a device, and a computer-readable storage medium. The method includes the following steps of: S11, receiving basic medical inquiry information; S13, performing sentence embedding processing on the basic medical inquiry information and converting it into a first digital vector; S15, performing matching processing in a large model based on the first digital vector to obtain first recommendation information, wherein the first recommendation information includes several first information items and a similarity degree of each first information item, and the several first information items are arranged in order of the similarity degree; S17, returning the first recommendation information; S21, receiving a selection of one or more first information items in the first recommendation information from a user; S23, optimizing sentence embedding processing of the basic medical inquiry information and training the large model based on selected first information items.

### TECHNICAL FIELD

The present disclosure relates to a large model-based medical information recommendation method, a device, and a computer-readable storage medium.

### BACKGROUND ART

Currently, medical diagnosis mainly relies on doctors' personal experience. If doctors lack experience, misdiagnosis or missed diagnosis may occur; to put it mildly, even if doctors have relatively rich experience, they may still misdiagnose, miss a diagnosis, or overlook some important steps due to certain factors. Therefore, there is an urgent need for a technical solution to reduce the associated risks.

### SUMMARY OF THE INVENTION

One objective of the present disclosure is to reduce the risks of doctors' misdiagnosis, missed diagnosis, or overlooking important information.

To this end, in a first aspect of the present disclosure, a large model-based medical information recommendation method is provided, including the following steps of: S11, receiving basic medical inquiry information; S13, performing sentence embedding processing on the basic medical inquiry information and converting it into a first digital vector; S15, performing matching processing in a large model based on the first digital vector to obtain first recommendation information, wherein the first recommendation information includes several first information items and a similarity degree of each first information item, and the several first information items are arranged in order of the similarity degree; S17, returning the first recommendation information; S21, receiving a selection of one or more first information items in the first recommendation information from a user.

In one embodiment of the present disclosure, after step S21, the method further includes S23, optimizing sentence embedding processing of the basic medical inquiry information and training the large model based on selected first information items.

In one embodiment of the present disclosure, the method further includes: S25, performing sentence embedding processing based on selected first information items and the basic medical inquiry information, converting them into a second digital vector; performing matching processing in the large model based on the second digital vector to obtain second recommendation information, wherein the second recommendation information includes several second information items and a similarity degree of each second information item, and the several second information items are arranged in order of the similarity degree; S27, returning the second recommendation information.

In one embodiment of the present disclosure, the method further includes: S33, optimizing sentence embedding processing of the basic medical inquiry information and training the large model based on selected second information items.

In one embodiment of the present disclosure, the method further includes: S41, receiving supplementary medical inquiry information; S43, performing sentence embedding processing on the basic medical inquiry information and the supplementary medical inquiry information, converting them into a third digital vector; S45, performing matching processing in the large model based on the third digital vector to obtain third recommendation information, wherein the third recommendation information includes several third information items and a similarity degree of each third information item, and the several third information items are arranged in order of the similarity degree; S47, returning the third recommendation information; S53, training the large model based on the basic medical inquiry information and the supplementary medical inquiry information.

In one embodiment of the present disclosure, step S53 includes training the large model using the basic medical inquiry information and the supplementary medical inquiry information of different patients.

In one embodiment of the present disclosure, the method further includes: S55, after training the large model based on the basic medical inquiry information and the supplementary medical inquiry information, providing early warning information, wherein the early warning information includes one or more of the following: drug allergy information; drug interaction information; important diagnostic information; important examination items; important consideration factors; high-risk symptom information.

In one embodiment of the present disclosure, the large model is stored on a local computer; the basic medical inquiry information includes several of the following items: gender; age; symptom information; physical sign information; preliminary examination results; past medical history.

In a second aspect of the present disclosure, a large model-based medical information recommendation device is provided, including: a receiving module for providing an information input interface and receiving basic medical inquiry information; a conversion module for performing sentence embedding processing on the basic medical inquiry information and converting it into a first digital vector; a large model, wherein the large model includes a large model database, a training module, and a return module; the training module performs matching processing in the large model database based on the first digital vector to obtain first recommendation information, wherein the first recommendation information includes several first information items and a similarity degree of each first information item, and the several first information items are arranged in order of the similarity degree; the return module returns the first recommendation information; wherein the receiving module is further for receiving a selection of one or more first information items in the first recommendation information from a user; the training module is further for optimizing sentence embedding processing of the basic medical inquiry information, training the large model, and updating the large model database based on selected first information items.

In one embodiment of the present disclosure, the training module is further for performing sentence embedding processing based on selected first information items and the basic medical inquiry information, converting them into a second digital vector; performing matching processing in the large model database based on the second digital vector to obtain second recommendation information, wherein the second recommendation information includes several second information items and a similarity degree of each second information item, and the several second information items are arranged in order of the similarity degree; optimizing sentence embedding processing of the basic medical inquiry information, training the large model, and updating the large model database based on selected second information items; the return module is further for returning the second recommendation information.

In a third aspect of the present disclosure, a computer-readable storage medium is provided, wherein the storage medium stores a computer program, and when executed by a processor, the computer program implements the method provided in the first aspect of the present disclosure.

In a fourth aspect of the present disclosure, an electronic apparatus is provided, including a memory, a processor, and a computer program stored in the memory and executable on the processor, wherein when the processor executes the program, the method provided in the first aspect of the present disclosure is implemented.

The present disclosure can match relatively complete medical information from patients' basic medical inquiry information based on a large model, compensates for the lack of experience of doctors, and reduces the risks of doctors' misdiagnosis, missed diagnosis, or overlooking key information.

### BRIEF DESCRIPTION OF THE DRAWINGS

FIG. 1 is a schematic diagram of a large model-based medical information recommendation method provided in a first embodiment of the present disclosure;

FIG. 2 is a schematic diagram of an interface for inputting basic medical inquiry information provided in a first embodiment of the present disclosure;

FIG. 3 is a schematic diagram of a process of a large model-based medical information recommendation method provided in a first embodiment of the present disclosure;

FIG. 4 is a schematic diagram of a process of an alternative solution for the medical information recommendation method shown in FIG. 3;

FIG. 5 is a schematic diagram of a process of a large model-based medical information recommendation method provided in a second embodiment of the present disclosure;

FIG. 6 is a schematic diagram of a large model-based medical information recommendation method for follow-up visits provided in a third embodiment of the present disclosure;

FIG. 7 is a schematic diagram of large model training and a large model-based medical information recommendation method based on the solution shown in FIG. 6;

FIG. 8 is a schematic diagram of a process of a large model-based medical information recommendation method provided in a third embodiment of the present disclosure;

FIG. 9 is a schematic diagram of large model optimization and adjustment for a large model-based medical information recommendation method provided in a fourth embodiment of the present disclosure; and

FIG. 10 is a schematic diagram of a large model-based medical information recommendation device provided in a fifth embodiment of the present disclosure.

### DETAILED DESCRIPTION OF THE INVENTION

The technical solutions in the embodiments of the present disclosure will be clearly and completely described below in conjunction with the accompanying drawings in the embodiments of the present disclosure. The described embodiments are merely a part rather than all of the embodiments of the present disclosure. All other embodiments obtained by those skilled in the art without making creative efforts based on the embodiments of the present disclosure shall fall within the protection scope of the present disclosure.

The terms “first”, “second”, etc., in the specification and claims of the present application are used to distinguish similar objects rather than to describe a specific order or sequence. It should be understood that the data used in this way can be interchanged under appropriate circumstances so that the embodiments of the present disclosure can be implemented in other orders than those illustrated.

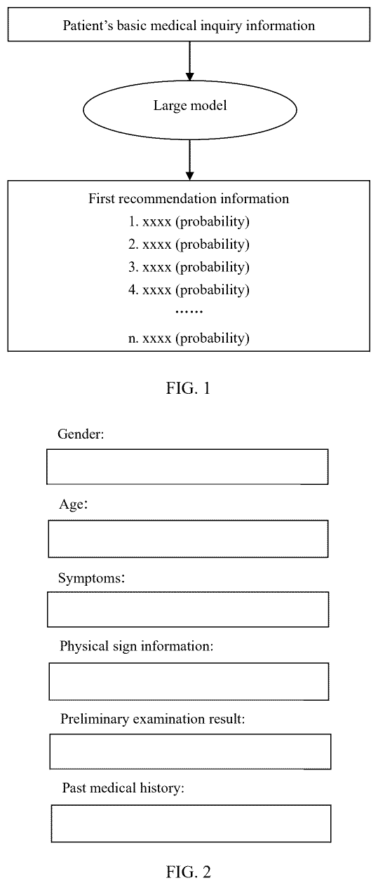

FIG. 1 is a schematic diagram of a large model-based medical information recommendation method provided in a first embodiment of the present disclosure, and FIG. 2 is a schematic diagram of an interface for inputting basic medical inquiry information provided in a first embodiment of the present disclosure. Referring to FIG. 1 and FIG. 2, in this embodiment, the patients' basic medical inquiry information includes several of the following items: gender; age; symptom information; physical sign information; preliminary examination results; past medical history. It should be understood that the present disclosure is not limited to the information items listed in this embodiment, but rather, more or fewer information items can be used as needed.

After the patients' basic medical inquiry information is obtained, first recommendation information including several first information items and a similarity degree of each first information item can be returned to a user, such as a doctor, through large model processing, and the several first information items are arranged in order of the similarity degree. In this embodiment, the first recommendation information is medical information associated with a patient's symptoms, such as the causes or treatment plans related to one or more symptoms. Preferably, the relevant information items are as complete as possible and are arranged from high to low based on probability or similarity degree.

In this way, the present disclosure can match relatively complete medical information from patients' basic medical inquiry information based on a large model, compensates for the lack of experience of doctors, and reduces the risks of doctors' misdiagnosis, missed diagnosis, or overlooking key information. Preferably, after a user, such as a doctor, selects a corresponding first information item from the first recommendation information, for example, after selecting or supplementing a corresponding diagnosis result or treatment plan, the relevant information is also used to optimize and train the large model to continuously improve the large model.

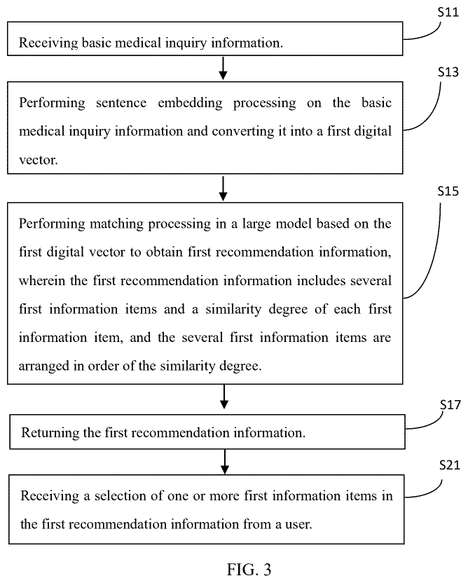

FIG. 3 is a schematic diagram of a process of a large model-based medical information recommendation method provided in a first embodiment of the present disclosure. Referring to FIG. 3, the method includes the following steps of:
S11, receiving basic medical inquiry information;
S13, performing sentence embedding processing on the basic medical inquiry information and converting it into a first digital vector;
S15, performing matching processing in a large model based on the first digital vector to obtain first recommendation information, wherein the first recommendation information includes several first information items and a similarity degree of each first information item, and the several first information items are arranged in order of the similarity degree;
S17, returning the first recommendation information;
S21, receiving a selection of one or more first information items in the first recommendation information from a user.

As mentioned above, the first recommendation information may be medical information associated with a patient's symptoms or basic medical inquiry information, such as the causes or treatment plans related to one or more symptoms. Preferably, the relevant information amount is as complete as possible and is arranged from high to low based on probability or similarity degree. Taking a 30-year-old male patient with diarrhea and vomiting as an example, in one embodiment, the large model returns relevant causes including viral gastroenteritis, infectious enteritis, inflammatory bowel disease, food poisoning, gastrointestinal infections, drug side effects, etc., and arranges them from high to low based on probability. Accordingly, this achieves the goal of assisting doctors in diagnosis and reducing the risks of doctors' misdiagnosis, missed diagnosis, or overlooking key information.

It should be understood that the first recommendation information may include relevant treatment plans, such as X-ray examination, MRI examination, CT examination, etc.

The aforementioned large model is preferably a large language model (LLM), one advantage of which is its ability to perform various natural language processing (NLP) tasks. Sentence embedding and conversion into digital vectors are existing processing methods in this field. For example, the basic information (i.e., the 1st, 2nd, and 6th items of information) of “a 50-year-old male with a healthy past medical history” can be converted into a digital vector [0.3, 0.2, 0.67, 1.2, 3.1 . . . ] after sentence processing, and may yield the following matching results:

$$\begin{matrix}Male + 50 + Healthy ( 0.9 ) ; \\ Male + 45 + Very Healthy ( 0.85 ) ; \\ Male + 49 + Good ( 0.77 ) ; \\ ……\end{matrix}$$

Where the values 0.9, 0.85, etc., in parentheses indicate probability.

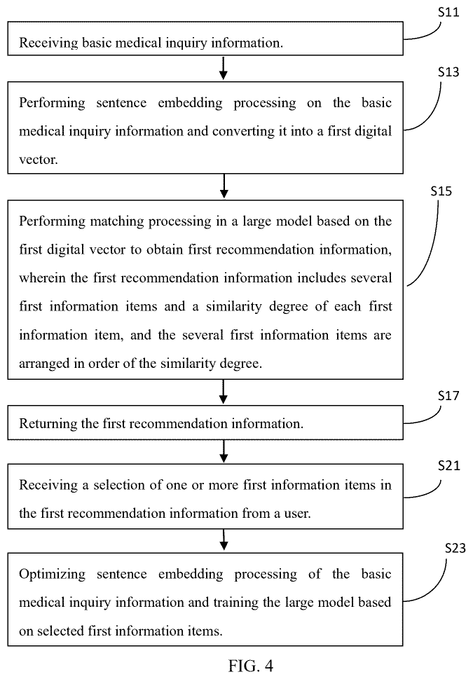

Referring to FIG. 4, as an alternative solution, following the process shown in FIG. 3, the process may further include step S23 as shown in FIG. 4: optimizing sentence embedding processing of the basic medical inquiry information and training the large model based on selected first information items. In this way, the quantity of training data can be further expanded, and the completeness and accuracy of the medical information recommendation method can be further improved. Here, “completeness” includes the completeness of each first information item in the first recommendation information, and “accuracy” includes the accuracy of the probability estimation and ranking of each first information item.

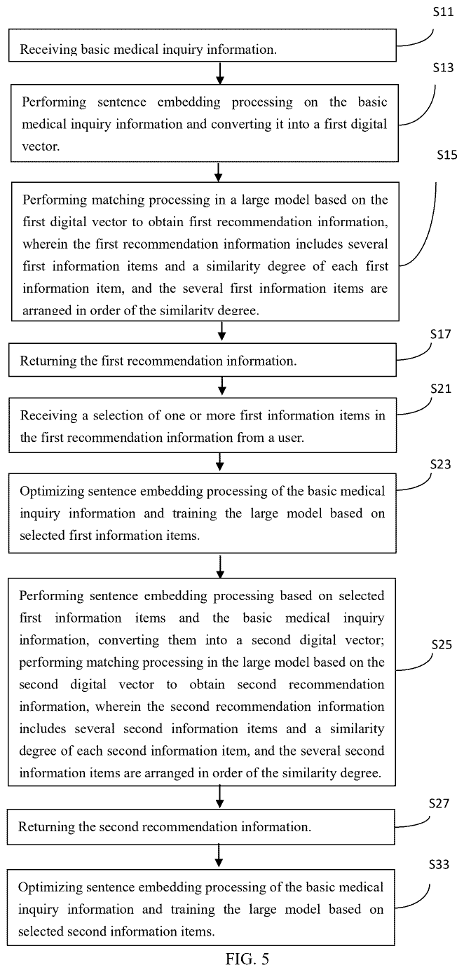

FIG. 5 is a schematic diagram of a process of a large model-based medical information recommendation method provided in a second embodiment of the present disclosure. The main difference between the second embodiment and the first embodiment shown in FIG. 3 lies in the addition of steps S25 and S27. In addition, as mentioned above, an optional step S23 may be added before step S25, and step S23 may be added after step S27. Wherein:
Step S25: performing sentence embedding processing based on selected first information items and the basic medical inquiry information, converting them into a second digital vector; performing matching processing in the large model based on the second digital vector to obtain second recommendation information, wherein the second recommendation information includes several second information items and a similarity degree of each second information item, and the several second information items are arranged in order of the similarity degree;
Step S27: returning the second recommendation information.

The advantage of this solution is that it can provide feedback to the large model according to the user's, such as a doctor's, selection of the first recommendation information, make relevant adjustments, and provide new recommendation information (i.e., second recommendation information). The information i items included in the second recommendation information may be the next examination plan, the next treatment plan, or new causes associated with the patient's symptoms or basic medical inquiry information, etc.

It should be understood that step S33 may be added after step S27: optimizing sentence embedding processing of the basic medical inquiry information and training the large model based on selected second information items. In this way, the quantity of training data can be further expanded, and the accuracy of the medical information recommendation method can be further improved. Here, “completeness” includes the completeness of each first information item in the first recommendation information and the completeness of each second information item in the second recommendation information; and “accuracy” includes the accuracy of the probability estimation and ranking of each first information item, as well as the accuracy of the probability estimation and ranking of each second information item.

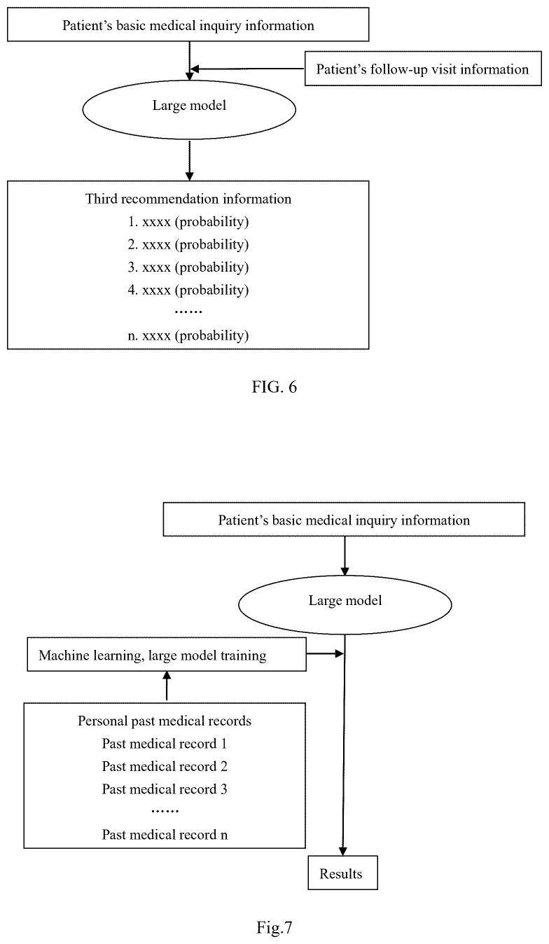

FIG. 6 is a schematic diagram of a large model-based medical information recommendation method for follow-up visits provided in a third embodiment of the present disclosure. As shown in FIG. 6, when a user returns for a follow-up visit, he or she may bring new medical inquiry information. After combining the follow-up medical inquiry information with the previous basic medical inquiry information, the large model can provide updated recommendation information (i.e., third recommendation information).

As shown in FIG. 7, it should be understood that machine learning and large model training can be performed based on the user's multiple medical inquiry experiences and past medical history to improve the accuracy and relevance of the system's recommendation information.

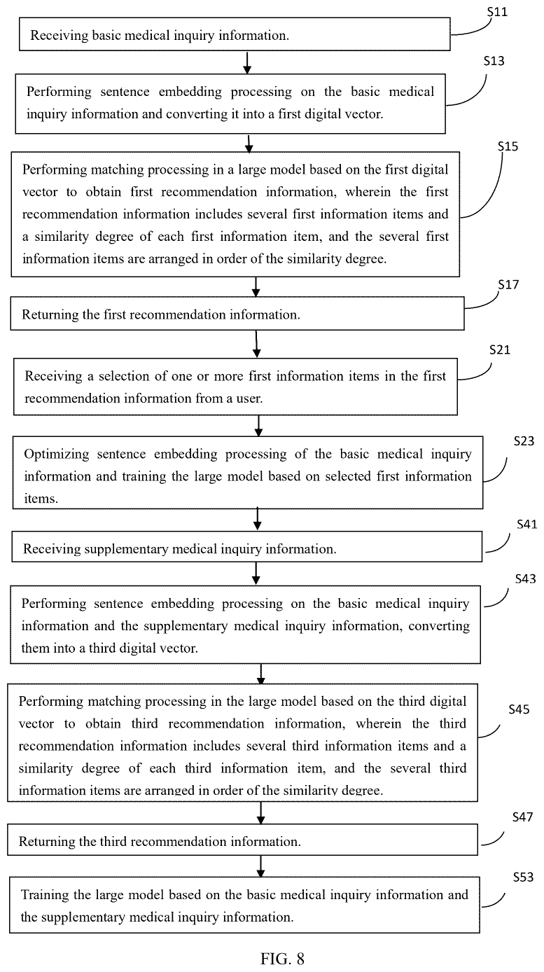

FIG. 8 is a schematic diagram of a process of a large model-based medical information recommendation method provided in a third embodiment of the present disclosure. As shown in FIG. 8, the main difference between the third embodiment and the first embodiment lies in the addition of steps S41, S43, S45, S47, and S53 after the optional step S23. Wherein:
Step S41: receiving supplementary medical inquiry information;
Step S43: performing sentence embedding processing on the basic medical inquiry information and the supplementary medical inquiry information, converting them into a third digital vector;
Step S45: performing matching processing in the large model based on the third digital vector to obtain third recommendation information, wherein the third recommendation information includes several third information items and a similarity degree of each third information item, and the several third information items are arranged in order of the similarity degree;
Step S47: returning the third recommendation information;
Step S53: training the large model based on the basic medical inquiry information and the supplementary medical inquiry information. Similarly, step S53 is an optional step. Adding step S53 can further expand the quantity of training data and further improve the accuracy of the medical information recommendation method. In this embodiment, “completeness” includes the completeness of each first information item in the first recommendation information and the completeness of each third information item in the third recommendation information; and “accuracy” includes the accuracy of the probability estimation and ranking of each first information item, as well as the accuracy of the probability estimation and ranking of each third information item.

The aforementioned supplementary medical inquiry information may be the medical inquiry information provided by the patient during a follow-up visit.

As relevant data about the patient continues to accumulate, each patient will have their own unique medical history. These data can be used for training models. Through machine learning, it is possible to learn what the normal daily state is and make early warnings if any abnormalities are detected. Such early warnings may include interactions between the drugs. For example, information about a patient's allergy to a certain drug is usually obtained from their medical history. However, if the patient uses multiple drugs during treatment, conflicts between the drugs can be prompted by the present disclosure. Therefore, the solutions shown in FIG. 7 and FIG. 8 can also be used for early warning. The provided the early warning information includes one or more of the following: drug allergy information; drug interaction information; important diagnostic information; important examination items; important consideration factors; high-risk symptom information.

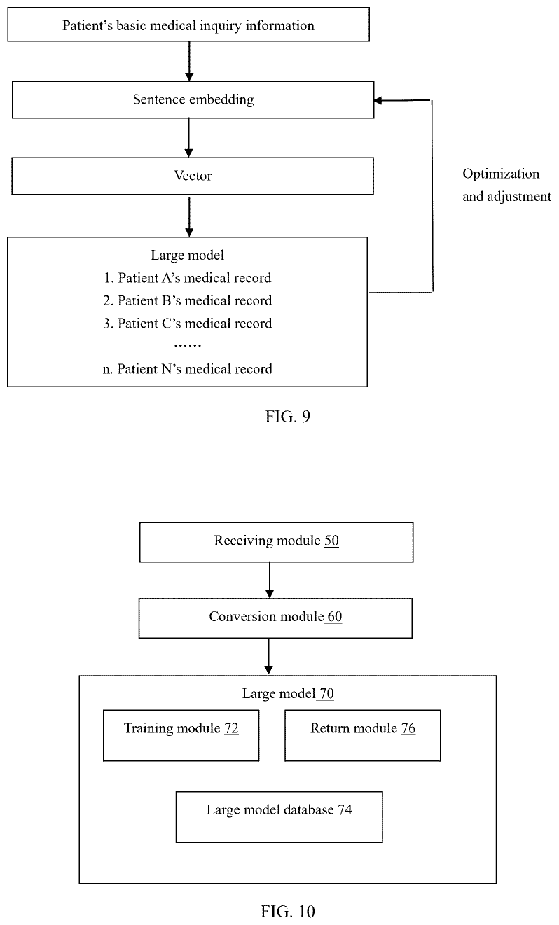

In addition to training the large model based on the longitudinal past medical history data of a certain patient, the large model can also be trained using the medical history data of a large number of patients. FIG. 9 is a schematic diagram of large model optimization and adjustment for a large model-based medical information recommendation method provided in a fourth embodiment of the present disclosure. As shown in FIG. 9, with the increasing of the number of patients and their medical records, the large model can perform self-learning, training, and self-optimization and adjustment of sentence embedding processing, thereby continuously improving the completeness and accuracy of information recommendations.

FIG. 10 is a schematic diagram of a large model-based medical information recommendation device provided in a fifth embodiment of the present disclosure. As shown in FIG. 10, a large model-based medical information recommendation device includes a receiving module 50, a conversion module 60, and a large model 70. The large model 70 includes a training module 72, a large model database 74, and a return module 76, wherein:
the receiving module 50 is for providing an information input interface and receiving basic medical inquiry information;
the conversion module 60 is for performing sentence embedding processing on the basic medical inquiry information and converting it into a first digital vector;
the training module 72 performs matching processing in the large model database 74 based on the first digital vector to obtain first recommendation information, wherein the first recommendation information includes several first information items and a similarity degree of each information item, and the several first information items are arranged in order of the similarity degree; the return module 76 returns the first recommendation information.

In addition, the receiving module 50 is further for receiving a selection of one or more first information items in the first recommendation information from a user; the training module 72, based on selected first information items, is further for optimizing sentence embedding processing of the basic medical inquiry information, training the large model, and updating the large model database 74, etc.

The above embodiments only express the preferred implementations of the present disclosure. Although the descriptions are relatively specific and detailed, they should not be understood as limiting the scope of the present invention patent. It should be noted that for persons skilled in the art, several modifications and improvements can still be made without departing from the concept of the present disclosure, such as combining different features from various embodiments, which all fall within the protection scope of the present disclosure.

1. A large model-based medical information recommendation method, comprising the following steps of:
S11, receiving basic medical inquiry information;
S13, performing sentence embedding processing on the basic medical inquiry information and converting it into a first digital vector;
S15, performing matching processing in a large model based on the first digital vector to obtain first recommendation information, wherein the first recommendation information includes several first information items and a similarity degree of each first information item, and the several first information items are arranged in order of the similarity degree;
S17, returning the first recommendation information;
S21, receiving a selection of one or more first information items in the first recommendation information from a user.

2. The medical information recommendation method according to claim 1, wherein after step S21, the method further comprises:
S23, optimizing sentence embedding processing of the basic medical inquiry information and training the large model based on selected first information items.

3. The medical information recommendation method according to claim 1, further comprising:
S25, performing sentence embedding processing based on selected first information items and the basic medical inquiry information, converting them into a second digital vector; performing matching processing in the large model based on the second digital vector to obtain second recommendation information, wherein the second recommendation information includes several second information items and a similarity degree of each second information item, and the several second information items are arranged in order of the similarity degree;
S27, returning the second recommendation information.

4. The medical information recommendation method according to claim 3,
further comprising:
S33, optimizing sentence embedding processing of the basic medical inquiry information and training the large model based on selected second information items.

5. The medical information recommendation method according to claim 1, further comprising:
S41, receiving supplementary medical inquiry information;
S43, performing sentence embedding processing on the basic medical inquiry information and the supplementary medical inquiry information, converting them into a third digital vector;
S45, performing matching processing in the large model based on the third digital vector to obtain third recommendation information, wherein the third recommendation information includes several third information items and a similarity degree of each third information item, and the several third information items are arranged in order of the similarity degree;
S47, returning the third recommendation information;
S53, training the large model based on the basic medical inquiry information and the supplementary medical inquiry information.

6. The medical information recommendation method according to claim 5, wherein step S53 includes training the large model using the basic medical inquiry information and the supplementary medical inquiry information of different patients.

7. The medical information recommendation method according to claim 5, further comprising:
S55, after training the large model based on the basic medical inquiry information and the supplementary medical inquiry information, providing early warning information, wherein the early warning information includes one or more of the following: drug allergy information; drug interaction information; important diagnostic information; important examination items; important consideration factors; high-risk symptom information;
the large model is stored on a local computer; the basic medical inquiry information includes several of the following items: gender; age; symptom information; physical sign information; preliminary examination results; past medical history.

8. A large model-based medical information recommendation device, comprising:
a receiving module for providing an information input interface and receiving basic medical inquiry information;
a conversion module for performing sentence embedding processing on the basic medical inquiry information and converting it into a first digital vector;
a large model, wherein the large model includes a large model database, a training module, and a return module; the training module performs matching processing in the large model database based on the first digital vector to obtain first recommendation information, wherein the first recommendation information includes several first information items and a similarity degree of each first information item, and the several first information items are arranged in order of the similarity degree; the return module returns the first recommendation information;
wherein the receiving module is further for receiving a selection of one or more first information items in the first recommendation information from a user; the training module is further for optimizing sentence embedding processing of the basic medical inquiry information, training the large model, and updating the large model database based on selected first information items.

9. The large model-based medical information recommendation device according to claim 8, wherein
the training module is further for performing sentence embedding processing based on selected first information items and the basic medical inquiry information, converting them into a second digital vector; performing matching processing in the large model database based on the second digital vector to obtain second recommendation information, wherein the second recommendation information includes several second information items and a similarity degree of each second information item, and the several second information items are arranged in order of the similarity degree; optimizing sentence embedding processing of the basic medical inquiry information, training the large model, and updating the large model database based on selected second information items;
the return module is further for returning the second recommendation information.

10. A computer-readable storage medium, wherein the storage medium stores a computer program, and when executed by a processor, the computer program implements the method according to claim 1.
